(19) United States
(12) Patent Application Publication
(10) Pub. No.: US 2016/0015438 A1
(43) Pub. Date: Jan. 21, 2016

(54) BONE ANCHOR DEVICE

(71) Applicants: Barron Douglas Elleby, Marietta, GA (US); Douglas Henry Elleby, Marietta, GA (US); Daniel Brian Lanois, Atlanta, GA (US)

(72) Inventors: Barron Douglas Elleby, Marietta, GA (US); Douglas Henry Elleby, Marietta, GA (US); Daniel Brian Lanois, Atlanta, GA (US)

(73) Assignee: PHALANX, LLC, Marietta, GA (US)

(21) Appl. No.: 14/334,235

(22) Filed: Jul. 17, 2014

(51) Int. Cl. A61B 17/86

(52) U.S. Cl. CPC A61B 17/861; A61B 17/864; A61B 17/863; A61B 2017/681

(57) ABSTRACT

A bone anchor device to connect two bone segments in a body has an elongated cylindrical member. The elongated cylindrical member has a shank, a first distal threaded end and a second proximal threaded end spaced by a middle portion of the shank. A first double helical thread formed on the first distal end has a pitch equal to or greater than a second helical thread formed at the second proximal end to create a compression between the two bone segments being connected. The shank has a maximum diameter extending the length of the second proximal thread end and extends therefrom on a constant tapered slope angle through the thread of the first distal end. This creates an increasing thread depth from a start of the distal threads to a maximum adjacent a tip at the distal end of the shank.

### TECHNICAL FIELD

The present invention relates to bone anchors generally, more specifically to bone anchors to connect two bone segments together providing a compressive force between the two segments. The bone anchor is ideally suited for bone fracture repair.

### BACKGROUND OF THE INVENTION

Bone anchors and/or bone screws are well known in the art and are adapted to connect fractured bones together bringing the fracture to a closed tight fitting relationship so that the bone segments can be closely connected hopefully in a compression type loading so that the fracture heals quickly and new bone growth occurs along the fracture line in a timely fashion. The inability to combine the bones tightly and to provide a secure compression of the bone fragments that have been fractured is an underlying problem that impedes the healing process.

The present invention as described hereinafter provides a unique way of bringing fractured bone segments together such that they are placed in compression, one against the other in a unique way using a simple single one piece device that allows the adjacent fractured components to be pulled together using one screw or anchor made according to the present invention.

### SUMMARY OF THE INVENTION

A bone anchor device to connect two bone segments in a body has an elongated cylindrical member. The elongated cylindrical member has a shank, a first distal threaded end and a second proximal threaded end spaced by a middle portion of the shank. A first double helical thread formed on the first distal end has a pitch greater than a second helical thread formed at the second proximal end to create a compression between the two bone segments being connected. The shank has a maximum diameter extending the length of the second proximal thread end and extends therefrom on a constant tapered slope angle through the thread of the first distal end. This creates an increasing thread depth from a start of the distal threads to a maximum adjacent a tip at the distal end of the shank. The threads of the first distal end and second distal end each have constant outside diameters. The outside diameter of threads of the first distal end is smaller than the outside diameter of the second proximal end. The shank has first zero degree slope angle along the length of the second proximal end and tapers thereafter on a second slope angle θ of 1 degree or more per side, preferably the second slope angle is between 2 and 4 degrees, yielding an inclusive tapered angle of 4 degrees to 8 degrees. Preferably, the shank second slope angle tapers on a constant slope angle through the first distal end.

The second proximal end has an aperture for receiving a fastener torqueing device. The second proximal end also has an outer surface having an acute angle relative to a plane perpendicular to an axis of the elongated member. The acute angle is 10 degrees to 30 degrees. Preferably, the acute angle is 15 degrees.

The device allows for a novel method of repairing a condition of fracture or alignment between two bone segments. The method has the steps of aligning and positioning two bone segments at a fracture or separation, optionally preparing a bone surface to create an inclined surface for entry of an elongated cylindrical member bone anchor in said bone segments; drilling a single insertion hole on an inclined angle or optionally perpendicular to a prepared inclined surface of a bone and through both of the bone segments; and inserting the bone anchor and tightening to rejoin and align the bone segments in compression by inserting the bone anchor having an acute angled outer surface at a proximal end to align flush with the outer bone surface or the inclined surface of the prepared bone segment. The prepared surface of the bone segments angle is inclined to correspond with the inclined end surface at a matching acute angle of the bone anchor proximal end so the outer surfaces are flush.

### BRIEF DESCRIPTION OF THE DRAWINGS

The invention will be described by way of example and with reference to the accompanying drawings in which:

FIG. 1 is a plan view of a bone anchor device made according to the present invention.

FIG. 1A is an end view showing the distal end of the bone anchor device of FIG. 1.

FIG. 1B is an end view showing the proximal end of the bone anchor device of FIG. 1.

FIG. 2 is a perspective view of the bone anchor device of FIG. 1 showing the proximal end.

FIG. 3 is a second perspective view of the bone anchor device of FIG. 1 showing the distal tip.

FIG. 4 is a cross sectional view of the first embodiment of the device taken along the line 4-4 of FIG. 1.

FIG. 5 is a plan view of a second embodiment of the invention.

FIG. 5A is an end view of the distal end of the second embodiment bone anchor device.

FIG. 5B is an end view of a proximal end of the bone anchor device of the second embodiment.

FIG. 6 is a perspective view showing the proximal end of the bone anchor device of the second embodiment.

FIG. 7 is a second perspective view showing the distal end of the bone anchor device of the second embodiment.

FIG. 8 is a cross sectional view of the second embodiment taken along line 8-8 of FIG. 5.

FIG. 9 is a plan view of a third embodiment according to the present invention.

FIG. 9A is an end view of the distal end of the third embodiment.

FIG. 9B is an end view of the proximal end of the third embodiment.

FIG. 10 is a first perspective view of the third embodiment showing the proximal end.

FIG. 11 is another perspective view showing the distal end of the third embodiment.

FIG. 12 is a cross sectional view of the third embodiment taken along line 12-12 of FIG. 9.

FIG. 13 is a fourth embodiment of the present invention shown in plan view.

FIG. 13A is an end view of the distal end of the fourth embodiment.

FIG. 13B is an end view of the proximal end of the fourth embodiment.

FIG. 14 is a perspective view showing the proximal end of the fourth embodiment.

FIG. 15 is a second perspective view showing the distal end of the fourth embodiment.

FIG. 16 is a cross sectional view taken along line 16-16 of FIG. 13 showing the fourth embodiment.

FIGS. 17A-17E are various views schematically showing a bone fracture and a method of predrilling a hole through two adjoining bone segments, FIG. 17C shows the bone anchor device of the present invention being cannulated and delivered through the drilled hole. FIG. 17D shows the device when being fully inserted. FIG. 17E has the device shown with the drill removed.

### DETAILED DESCRIPTION OF THE INVENTION

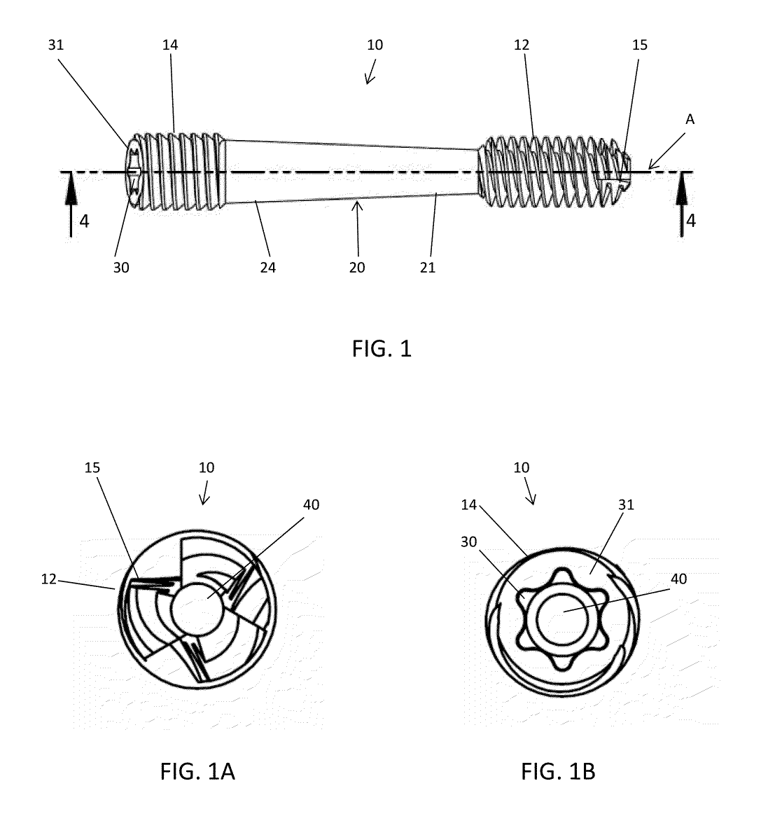

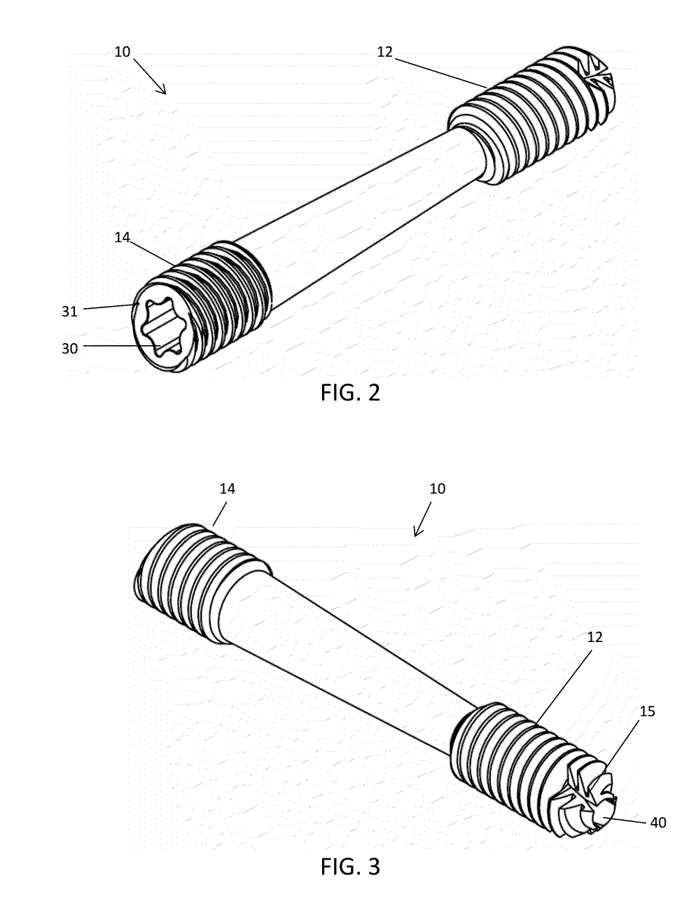

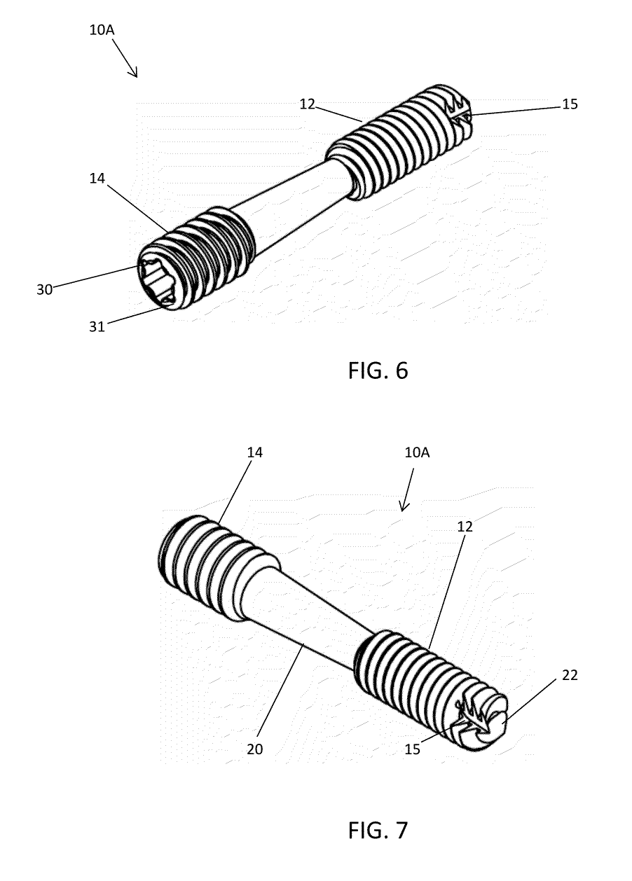

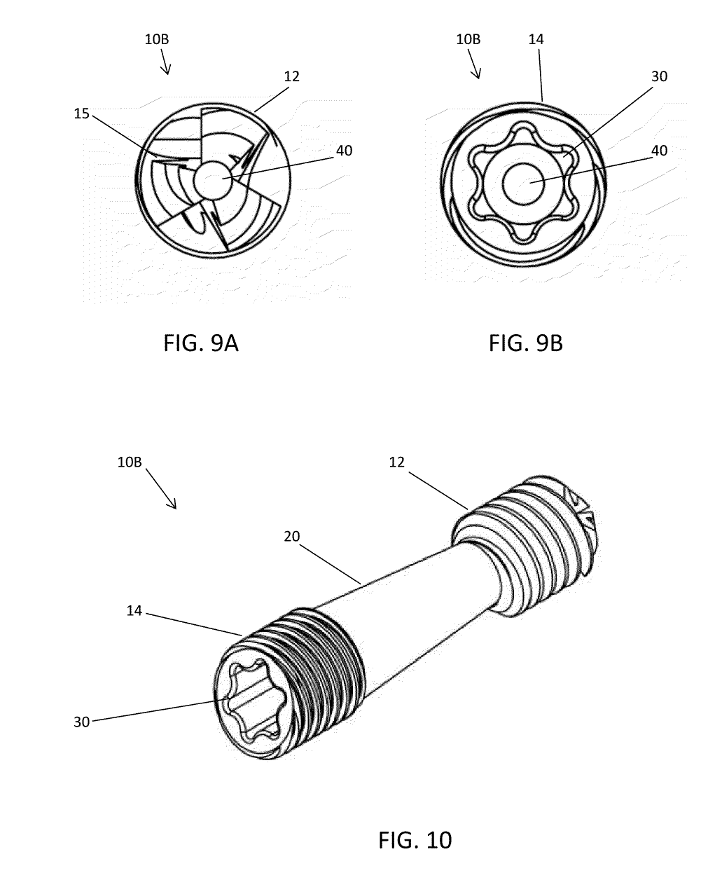

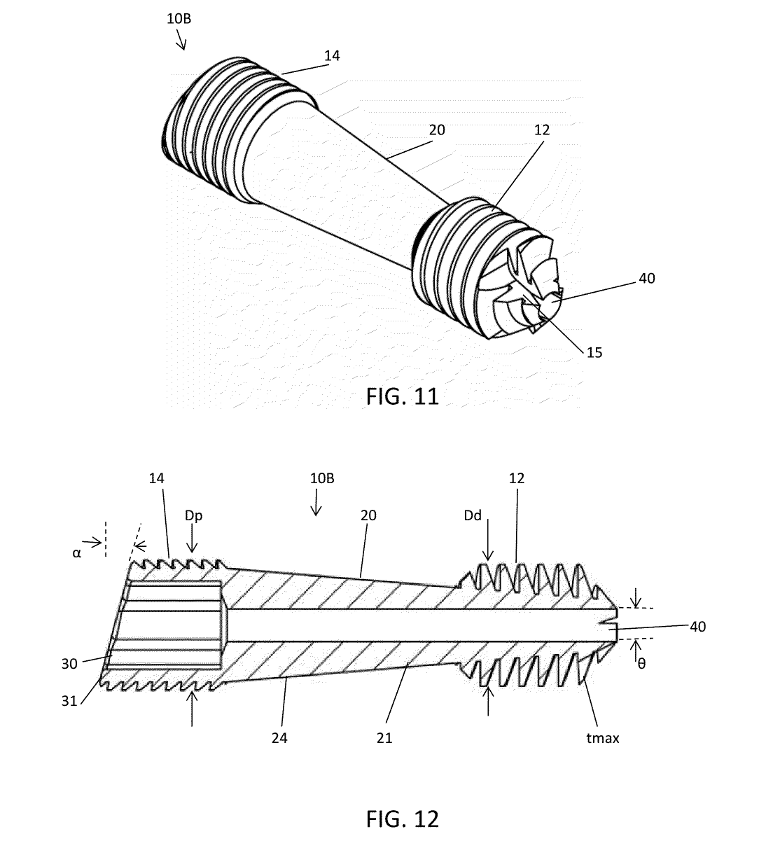

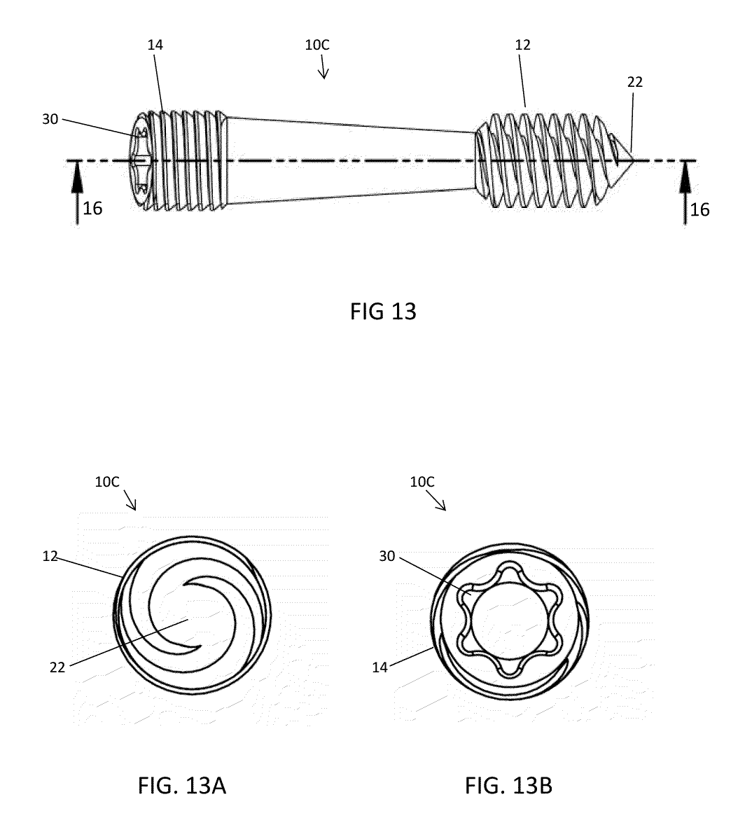

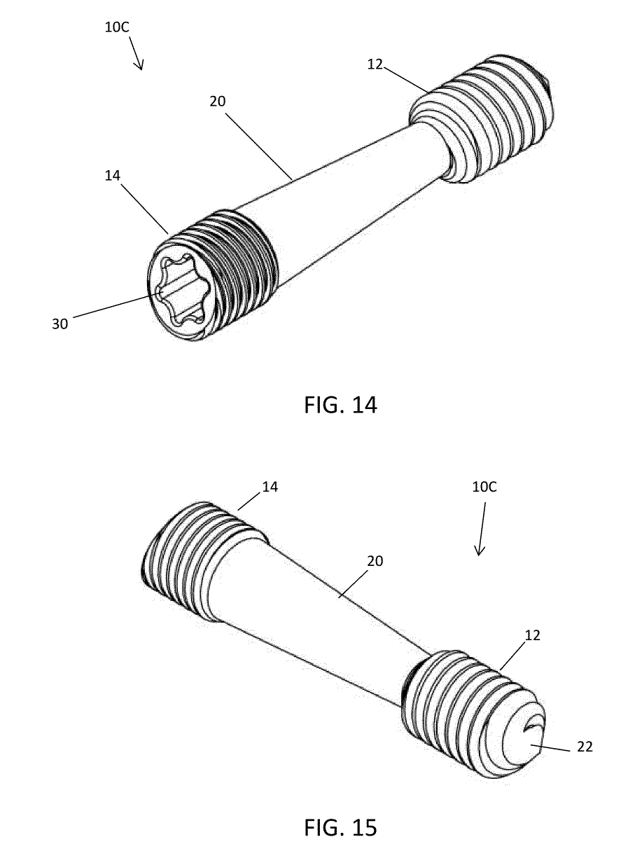

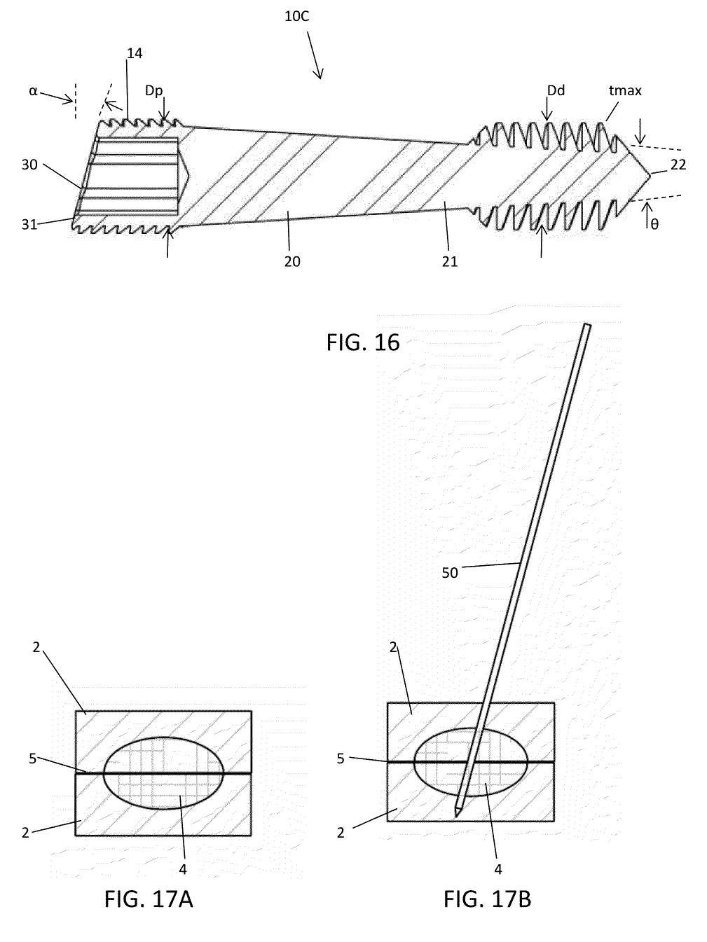

With reference to FIGS. 1-16, various views of the exemplary bone anchor devices made according to the present invention are illustrated. In FIGS. 1-4, a first embodiment of the bone anchor 10 is illustrated wherein it is cannulated. In FIGS. 5-8, a second embodiment is illustrated wherein the bone anchor 10A is solid. In FIGS. 9-12, a third embodiment bone anchor 10B is shown wherein the bone anchor 10B is cannulated and is similar to the bone anchor 10 illustrated in FIGS. 1-4, however of a different size. FIGS. 13-16 show a fourth embodiment of the invention, bone anchor 10C illustrating the bone anchor in a solid configuration similar to the second embodiment 10A. All of the embodiments have features that are common and throughout the specification, those common features are identified by similar reference numerals.

With reference to a first embodiment of the invention as illustrated in FIG. 1, the bone anchor 10 is illustrated having an elongated member 20 having a shank 21, a first distal threaded end 12 and a second proximal threaded end 14 spaced by a middle portion 24 of the shank 21. As shown in FIG. 1A, the distal end is illustrated wherein a hole or center opening 40 is provided that extends throughout the elongated member 20. The leading edges of the first distal threaded end 12 has cutting edges 15 that help cut threads into the bone segments into which the bone anchor 10 will be threaded. FIG. 1B shows the proximal end 14 of the bone anchor 10. The proximal end 14 has an aperture 30 configured to receive a driving element to rotate the bone anchor 10 into two adjacent bone segments when being attached to repair a fracture. As shown, the opening 40 extends throughout the bone anchor 10 in this embodiment. Thus the bone anchor 10 as illustrated is cannulated. As shown in this exemplary embodiment, the bone anchor 10 forms a compression screw having a size of 3.0 mm×20 mm. These sizes can be varied both in length and in diameters. As shown, the size reflecting the diameter is established at the proximal end 14 of the bone anchor 10.

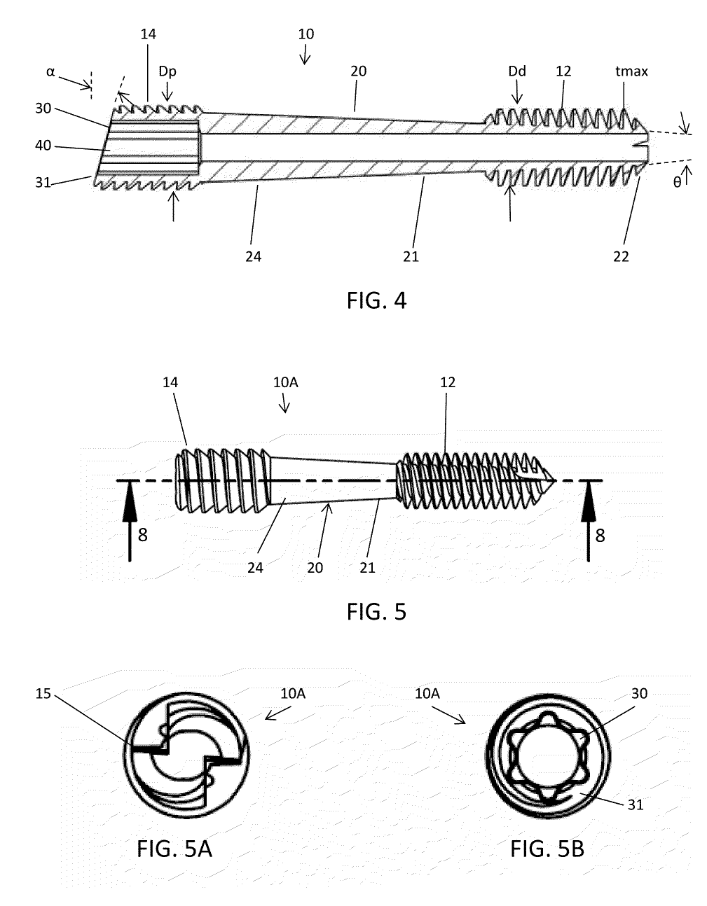

This sizing of the bone anchor is best illustrated in FIG. 4 wherein the bone anchor 10 has the elongated member 20 extending the entire length of the bone anchor 10. The middle shank portion 24 is smooth and extends to ends. At one end is the first distal threaded end 12 and on the opposite end is the second proximal threaded end 14. As used herein, distal means furthest end into the bone and proximal is the closest end to the surgeon installing the bone anchor. As shown, the proximal end 14 threads are uniform in diameter having a maximum diameter Dp. An end surface 31 of the proximal end 14 is slanted on an angle α. The angle α creates a slant preferably between 15 and 30 degrees. This angle α is designed so that the bone anchor 10 when threaded into a bone segment on a similar mating angle can be aligned so that the proximal end 14 upon being threaded into the bone segments can be positioned so that the outer surface 31 is flush with the outer surface of the bone. A further feature of the present invention is that the elongated member 20 at the proximal end 14 has a zero slope throughout the proximal threads so that the threads are of a constant thickness or pitch. As illustrated, in the middle portion 24 where the shank 21 is smooth, the shank 21 tapers on a constant angle θ. This constant angle θ extends not only through the middle portion 24 as illustrated of the shank 21, but also extends through to the tip 22. This angle θ ensures that the distal threads diameter Dd can be maintained constant, however, as the threads approach the tip 22, the thread engagement surfaces increase in depth on a continually increasing basis to a maximum tmax as shown adjacent the tip 22. This feature is quite unique in that it enables the bone anchor 10 at the farthest end of the device to have an increasing engagement of thread to bone at the distal end 12. This assists in ensuring that as the thread is driven into the bone and particularly into the cortical bone, the opposite end of the threads closest to the tip 22 will be engaged with an increasing thickness of distal threads helping to assist in building a maximum amount of bone contact for compression. As shown, the distal threads are formed as a first double helical thread having a pitch equal to or greater than the second helical thread formed at the second proximal end 14. This variation of the pitch of the helical threads ensures that as the bone anchor 10 is driven into the bone, its bone engagement at one end is increasing faster than at the proximal end 14. In so doing, this enables the bone segments to be drawn together quickly closing any gaps from a fracture in a tight compressive load as the distal end 12 advances more rapidly into the bone than the proximal end 14 ensuring that the two bone segments at a fracture can be drawn together tightly as the bone anchor 10 is inserted into the segments.

Referring back to FIG. 1A, as illustrated the cutting edges 15 are shown so that 3 equally disposed cutting edges 15 are created on angles of approximately 120 degrees relative to the other to assist in having the distal end 12 penetrate into the bone segments.

With reference to FIGS. 5-8, a second embodiment of the invention is illustrated, 10A. The embodiment 10A is similar to the embodiment illustrated in FIG. 1; however the proximal end 14 and the distal end 12 are spaced by a shorter middle portion 24 wherein the entire elongated member 20 is of a different size. As illustrated the size of this exemplary bone anchor or compression screw 10A is 2.0 mm by 10 mm. Again, the sizes can vary as a matter of design choice. However, the length and diameter of this particular embodiment is of a size to enable one to appreciate that this is a relatively small device adapted to fit into reasonably small fractures to bring the two bone segments together. As shown, the device 10A has the distal end 12 with two cutting edges 15 in this embodiment 10A, also it has the same aperture 30 for receiving a tool to torque the device 10A into the bone segments. As further shown, in FIG. 7, the tip 22 is illustrated wherein the distal end 12 has a sharp pointed tip 22 that is solid therefore the entire elongated member 20 unlike the first embodiment has a solid shank. A second difference is that the proximal end 14 is not inclined in this embodiment which is an optional feature. This embodiment is designed to be entered into a bone relatively transversely or perpendicularly to the surface such that, when driven into the bone segments, the flat outer surface or edge 31 will maintain a smooth relationship with the bone segments so that it can be buried flush. Alternatively, the angle a could be provided in this embodiment if so desired.

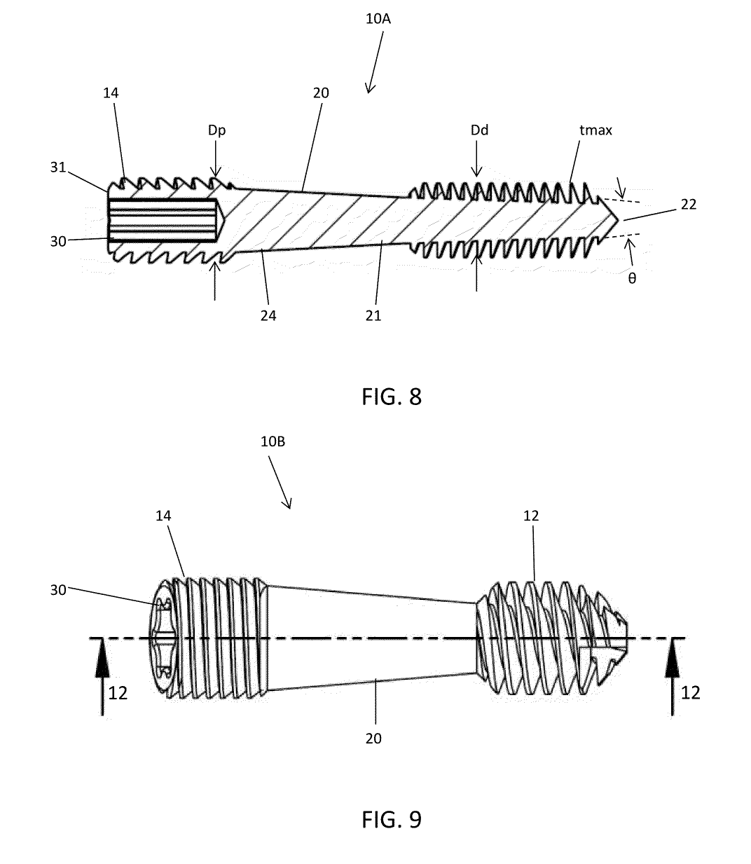

With reference to FIG. 8, a cross sectional view is shown wherein the shank 21 at the middle portion 24 extends on a constant angle θ from the threads at the proximal end 14 through the threads at the distal end 12 adjacent the tip 22. As shown, this constant angle θ allows the distal threads at the distal end 12 to have a maximum tmax adjacent the tip 22 as previously discussed. Similarly the diameter Dd at the distal end 12 is slightly smaller than the diameter Dp at the proximal end 14. The diameter Dp as illustrated is constant and uniform across the threads at the proximal end 14 as is the diameter Dd at the distal end 12, very similar to the first embodiment 10 having the increasing thread to bone engagement feature.

With reference to FIGS. 10-12, a third embodiment 10B of the present invention is shown. This bone anchor device 10B has all the features illustrated in the first embodiment and is virtually identical in that regard. However, it is made of a size 4.0 mm×16 mm. Again, the 4 mm size is measured at the threads of the proximal end 14. As illustrated, this exemplary size illustrates a very large diameter can be created and that the shank 21 of the elongated member 20 is truncated in such a fashion that it provides for a very short screw with a much higher constant slope angle θ. The angle θ in this embodiment is substantially increased relative to the first embodiment. As such, tmax adjacent the tip 22 has a large amount of surface thread engagement area for securing the bone segments, otherwise it is virtually identical to the first embodiment device 10 in the earlier description of the invention.

With reference to FIGS. 13-16, a fourth embodiment exemplary bone anchor 10C of the invention is illustrated wherein the bone anchor device 10C is made solid having a configuration virtually identical to that of the second embodiment 10A. however, in this embodiment, the optional sloped proximal end 14 at the end surface 31 has the inclination a such that the end 31 is inclined 10 degrees or more. This inclination enables the bone anchor 10C as previously discussed to be inserted either on an incline or on a prepared surface that has been inclined such that when the bone anchor 10C is drilled into the two bone segments the outer end surface 31 will remain flush with an outer surface of the bone segment to which the proximal end 14 is engaged. Similar to the other embodiments, the distal end 12 has a diameter Dd with a maximum thread tmax adjacent the tip 22. As shown, the shank 21 similarly has a constant slope angle θ from the proximal threads 14 extending all the way through the distal threads 12 on a slope angle of approximately 5 degrees or more forming an angle θ. And as in all the embodiments the distal threads 12 have a double helix such that they advance into the bone segments faster and as they advance it is important to note that the initial thread adjacent the tip 22 has a maximum bone engagement tmax such that as it moves into the cortical bone region, it engages more bone surface allowing for a tighter compression to be achieved between the bone segments being repaired. In this fourth embodiment 10C, the exemplary size of the bone anchor 10C is 4.0 mm×20 mm.

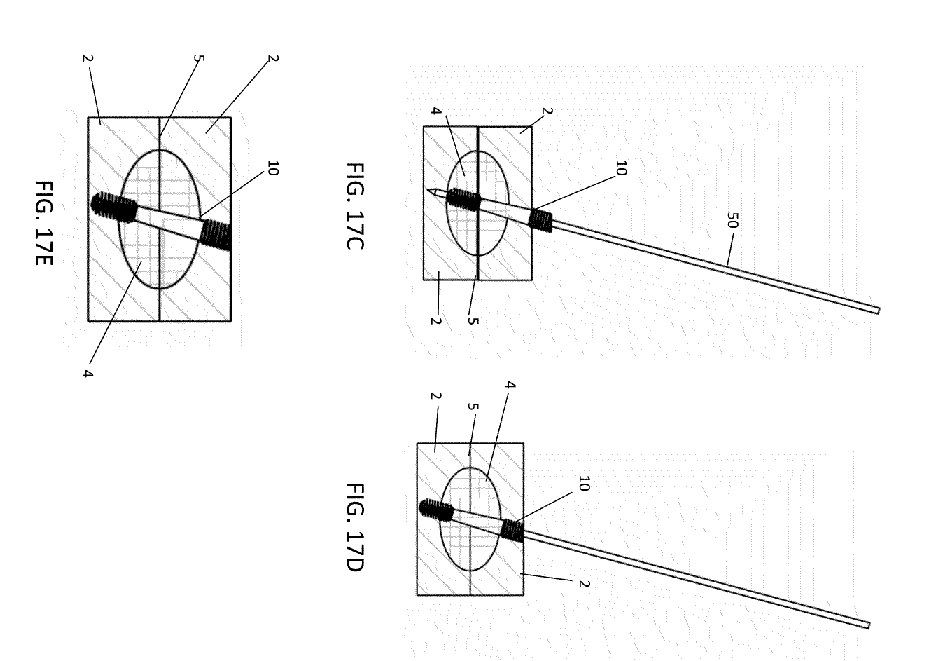

With reference to FIGS. 17A-17E, a schematic illustration is provided wherein bone segments 2 are shown with a fracture 5. The bone segments 2 have a center cancellous bone region 4 surrounded by the cortical bone regions 2. As illustrated, a guide wire 50 can be driven into the bone segments 2 through the cortical bone as illustrated crossing the fracture 5 to the opposite segment 2. At this point, a cannulated bone anchor device 10 or 10B can be slipped over the guide wire 50 and driven by a hollow driving tool slid over the guide wire 50 and fitted into the aperture 30 into the bone segments 2 (not illustrated is the driving tool), however as the bone anchor 10, 10B is torqued into the bone 2, 4 along the guide wire 50. As this occurs, the segments 2 are drawn closer together creating a tighter compacted compression against the two bone segments 2 along the fracture line 5. Once the bone anchor 10, 10B is fully driven into the bone segments 2, the outer surface 31 will be flush as illustrated in FIG. 17D. At this point the guide wire 50 can be removed from the bone segments 2 and the repair will have been made. What is important to understand is that the distal end 12 of the bone anchor 10 will be advancing more rapidly thereby due to its double helix configuration as such as it enters into the lower bone segment 2 the proximal end 14 will be engaging the upper cortical bone 2 and as they are being torqued, the segments 2 are being pulled together very tightly. This creates a unique configuration wherein the bone anchor 10 is shown being provided on an inclination in this embodiment. However, as previously discussed, the bone anchor could enter transversely and have a flat surface as is illustrated in the second embodiment 10A.

It is appreciated that the embodiments wherein the bone anchor is solid can be inserted by predrilling the two bone segments creating an opening wherein the bone anchor 10A and 10C can then be driven into the segments 2 without the use of the guide wire 50 which is conveniently usable in the cannulated versions of the bone anchor 10 and 10B, but not needed in the solid versions 10A and 10C.

While the bone anchors 10 are shown in various sizes and dimensions it is also important to note that the materials can vary. These bone anchors can be made of any suitable implantable metal material such as titanium and/or stainless steel or alternatively can be formed out of plastic materials such as PEEK (polyether ether ketone). While the embodiments are shown in particular sizes, it is appreciated that the sizes can vary, it is also appreciated that while the bone anchor is shown having certain features, some alterations can be made. However, it is important that the bone anchors maintain the general configuration as shown to achieve the maximum compression possible while also providing preferably a smooth surface where the bone anchor is exposed at the proximal end surface 31.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

What is claimed is:

1. A bone anchor device to connect two bone segments in a body comprising:
an elongated cylindrical member having a shank, a first distal threaded end and a second proximal threaded end spaced by a middle portion of the shank;
a first double helical thread formed on the first distal end having a pitch equal to or greater than a second helical thread formed at the second proximal end to create a compression between the two bone segments being connected; and
wherein the shank has a maximum diameter extending the length of the second proximal thread end and extends therefrom on a constant tapered slope angle through the thread of the first distal end creating an increasing thread depth from a start of the distal threads to a maximum adjacent a tip at the distal end of the shank, and wherein the threads of the first distal end and second proximal end each have constant outside diameters.

2. The bone anchor device of claim 1 wherein the outside diameter of threads of the first distal end is smaller than the outside diameter of the second proximal end.

3. The bone anchor device of claim 1 wherein the shank has first zero degree slope angle along the length of the second proximal end and tapers thereafter on a second slope angle of 1 degree or more.

4. The bone anchor device of claim 3 wherein the second slope angle is 10 degrees or more.

5. The bone anchor device of claim 3 wherein the shank tapers on a constant slope angle through the first distal end.

6. The bone anchor device of claim 4 wherein the slope tapers on a constant slope angle through the first distal end.

7. The bone anchor device of claim 1 wherein the second proximal end has an aperture for receiving a fastener torqueing device.

8. The bone anchor device of claim 1 wherein the elongated member is cannulated having a center opening through the shank to receive a guide wire.

9. The bone anchor device of claim 1 wherein the shank of the elongated member is solid.

10. The bone anchor device of claim 4 wherein the second proximal end has an outer surface having an acute angle α relative to a plane perpendicular to an axis of the elongated member.

11. The bone anchor device of claim 10 wherein the acute angle α is 10 degrees to 30 degrees.

12. The bone anchor device of claim 10 wherein the acute angle α is 15 degrees.

13. A bone anchor device to connect two bone segments in a body comprising:
an elongated cylindrical member having a shank, a first threaded distal end and a second proximal threaded end spaced by a middle portion of the shank;
a first double helical thread formed on the first distal end having a pitch equal to or greater than a second helical thread formed at the second proximal end to create a compression between the two bone segments being connected; and
wherein the second proximal end has an outer surface having an acute angle relative to a plane perpendicular to an axis of the elongated member.

14. The bone anchor device of claim 13 wherein the shank has a maximum diameter extending the length of the second proximal thread end and extends therefrom on a constant tapered slope angle through the thread of the first distal end creating an increasing thread depth from a start of the distal threads to a maximum adjacent a tip at the distal end of the shank, and wherein the threads of the first distal end and second distal end each have constant outside diameters

15. The bone anchor device of claim 14 wherein the second proximal end has an aperture extending inward from the outer surface parallel to the axis for receiving a fastener torqueing device.

16. The bone anchor device of claim 14 wherein the outside diameter of threads of the first distal end are smaller than the outside diameter of the second proximal end.

17. The bone anchor device of claim 13 wherein the shank has first zero degree slope angle along the length of the second proximal end and tapers thereafter on a second slope angle of 1 degree or more.

18. The bone anchor device of claim 13 wherein the second slope angle is 10 degrees or more.

19. The bone anchor device of claim 13 wherein the shank tapers on a constant slope angle through the first distal end.

20. The bone anchor device of claim 13 wherein the slope tapers on a constant slope angle through the first distal end.

21. A method of repairing a condition of fracture or alignment between two bone segments comprises:
aligning and positioning two bone segments at a fracture or separation, optionally preparing a bone surface to create an inclined surface for entry of an elongated cylindrical member bone anchor in said bone segments;
forming a single insertion hole on an inclined angle or optionally perpendicular to a prepared inclined surface of a bone and through both of the bone segments; and
inserting the bone anchor and tightening to rejoin and align the bone segments in compression by inserting the bone anchor having an acute angled outer surface at a proximal end to align flush with the outer bone surface or the inclined surface of the prepared bone segment.

22. The method of claim 21 wherein the prepared surface of the bone segments angle is inclined to correspond with the inclined end surface at a matching acute angle.

23. The method of claim 21 wherein the step of forming the insertion hole includes placing a guide wire into the bone segments and placing the bone anchor onto the guide wire.

24. The method of claim 21 wherein the step of forming the hole includes drilling.
